(19) United States
(12) Patent Application Publication
(10) Pub. No.: US 2026/0090870 A1
(43) Pub. Date: Apr. 2, 2026

(54) URINARY INCONTINENCE CLAMP DEVICE FOR MEN

(71) Applicant: MEDICARETEC CO., LTD., Seoul (KR)

(72) Inventor: Han Yong CHUN, Seoul (KR)

(21) Appl. No.: 19/347,664

(22) Filed: Oct. 1, 2025

Related U.S. Application Data
(63) Continuation of application No. PCT/KR2024/004975, filed on Apr. 12, 2024.

(30) Foreign Application Priority Data
Apr. 12, 2023 (KR) 10-2023-0048465
Apr. 12, 2024 (KR) 10-2024-0049594

(51) Int. Cl. A61F 2/00

(52) U.S. Cl. CPC A61F 2/0018; A61F 2/0054

(57) ABSTRACT

The present invention relates to a urinary incontinence clamp device for men utilizing magnetic force and, more particularly, to a urinary incontinence clamp device for men characterized in that, as a second clamp unit slides along an inner surface of a first clamp unit, the penis is gradually secured by a penile fixing portion, whereby the force applied to the penis is gradually exerted, reducing impact force applied to the penis, and thereby improving wearing comfort by preventing pain in the penis during fixation.

### TECHNICAL FIELD

The present invention relates to a urinary incontinence clamp for men, and more particularly, to a male urinary incontinence clamp comprising: a first clamp unit comprising an inner surface formed in a ring shape and comprising a first penile fixing portion; and a second clamp unit comprising an outer surface and comprising a handle portion and a second penile fixing portion, the clamp being configured to appropriately adjust a compressive force applied to the urethra.

### BACKGROUND ART

Urinary incontinence refers to a condition in which urine is discharged regardless of a person's voluntary control. In recent years, as the average life expectancy has increased and the elderly population has grown, the incidence of urinary incontinence has been on the rise. Types of urinary incontinence include: true urinary incontinence, in which urine is discharged without any triggering factor; stress urinary incontinence, which occurs when abdominal pressure increases due to sneezing, coughing, or the like; urge urinary incontinence, in which urination cannot be voluntarily delayed when the urge to void arises; and overflow urinary incontinence, which occurs when the bladder becomes overfilled and urine leaks involuntarily.

Particularly, with the increase in the elderly population and the westernization of dietary habits, the incidence of prostate-related diseases, such as benign prostatic hyperplasia and prostate cancer, has been rising, and the incidence rate of urinary incontinence following treatment for such diseases reaches 5 to 45 percent.

Male stress urinary incontinence, which occurs as a result of sphincter dysfunction following treatment for prostate diseases as described above, causes significant discomfort to patients. Accordingly, efforts have been made to enable patients to maintain daily activities by using diapers, pads, condom catheters, or the like.

Accordingly, in order to prevent urinary incontinence that occurs during daily activities regardless of a person's voluntary control as described above, urinary incontinence prevention devices or apparatuses have been developed and are in use.

Conventional urinary incontinence prevention devices focus primarily on compressing the urethra to prevent leakage. Consequently, these devices are often bulky, do not properly accommodate variations in penile size or wearing conditions, and apply continuous excessive pressure, which can lead to discomfort and, in severe cases, penile necrosis.

In addition, in conventional urinary incontinence prevention devices, when worn, ventilation is insufficient, which may cause skin diseases around the penis and may also lead to hygiene-related problems.

Furthermore, conventional urinary incontinence prevention devices have primarily employed a method of compressing the urethra of the penis by utilizing an elastic force generated through contraction of an elastic member made of a rubber material. However, as the contraction and relaxation of the elastic member are repeated, the elastic force deteriorates, resulting in insufficient compression of the urethra. In addition, over time, the rubber material may wear out or degrade and eventually break, rendering the device unusable. In such cases, inconveniences may arise in that the elastic member made of the rubber material must be replaced.

Accordingly, there is a need for the development of a male urinary incontinence prevention device (clamp) capable of adjusting the compressive force applied to the penis from the standpoint of the urinary incontinence prevention function, improving ventilation performance, and, from the standpoint of device durability, preventing wear over time so that the service life of the device can be semi-permanently extended.

### DISCLOSURE

### Technical Problems

The technical problem to be solved by the present invention is to provide a male urinary incontinence clamp device that can be easily worn and removed by users, is minimized in size, is capable of adjusting the compressive force applied to the penis, and has improved ventilation performance.

### Technical Solution

In order to achieve the above object, the present invention provides a male urinary incontinence clamp comprising: a first clamp unit having an inner surface formed in a ring shape and including a first penile fixing portion; and a second clamp unit having an outer surface and including a handle portion and a second penile fixing portion, wherein the outer surface of the second clamp unit is brought into close contact with and fixed to the inner surface of the first clamp unit, the second clamp unit is slidable along the inner surface of the first clamp unit, and the penis is secured between the first penile fixing portion and the second penile fixing portion by utilizing magnetic force.

In addition, the first clamp unit may comprise a first magnetic portion, the second clamp unit may comprise a second magnetic portion, and the first magnetic portion and the second magnetic portion may be arranged to generate an attractive force toward each other.

In addition, the inner surface of the first clamp unit may comprise one or more first retaining members, and the outer surface of the second clamp unit may comprise one or more second retaining members that are structurally corresponding to the one or more first retaining members.

In addition, the first magnetic portion may comprise one or more first retaining members, and the second magnetic portion may comprise one or more second retaining members that are structurally corresponding to the one or more first retaining members.

In addition, a surface of the first penile fixing portion or the second penile fixing portion may be made of an elastic material, and the surface may be formed with a plurality of protrusions made of the elastic material.

In addition, the plurality of protrusions may have a width ranging from 0.05 to 1.0 times a length in a z-axis direction.

In addition, the plurality of protrusions may project from the surface in a direction along the x-axis or opposite to the x-axis direction by 0.1 to 2.0 mm.

In addition, the elastic material may be an elastic material comprising silicone.

In addition, the elastic material comprising silicone may be characterized by a mixture of silicone and a rubber material, wherein the rubber material may be one or more selected from the group consisting of acrylonitrile butadiene rubber, styrene-butadiene rubber, natural rubber, polyvinyl chloride, polyethylene, ethylene-vinyl acetate, latex, silicone rubber, styrene-isoprene-styrene copolymer, styrene-ethylene-butylene-styrene copolymer, polyvinyl alcohol, butyl rubber, chloroprene rubber, and polyurethane.

### ADVANTAGEOUS EFFECTS

The present invention relates to a urinary incontinence clamp device for men, which can be easily worn and removed by users, can mitigate an impact force applied to the penis when worn, can allow adjustment of a sense of compression of the penis even in a worn state, and can improve ventilation performance.

### BRIEF DESCRIPTION OF DRAWINGS

FIGS. 1A and 1B illustrate an operating state of a male urinary incontinence clamp according to an embodiment of the present invention.

FIGS. 2A and 2B illustrate a plurality of first retaining members and a plurality of second retaining members located on a first clamp unit and a second clamp unit, respectively, according to an embodiment of the present invention.

FIGS. 3A and 3B illustrate an operating state of a plurality of first retaining members and a plurality of second retaining members according to an embodiment of the present invention.

FIGS. 4A and 4B illustrate a single first retaining member and a single second retaining member located on a first clamp unit and a second clamp unit, respectively, according to an embodiment of the present invention.

FIGS. 5A and 5B illustrate an operating state of a single first retaining member and a single second retaining member according to an embodiment of the present invention.

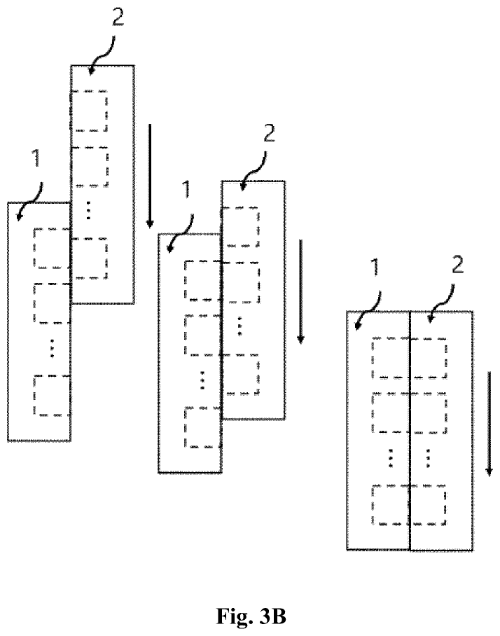

FIG. 6A illustrates, in a three-dimensional view, a plurality of protrusions formed on a U-shaped second penile fixing portion (221) in an x-axis direction, a y-axis direction, and a z-axis direction.

FIG. 6B illustrates a plurality of protrusions formed in an x-axis direction on a surface of a U-shaped second penile fixing portion (221), as viewed from an opposite z-axis direction.

FIG. 6C illustrates, in a three-dimensional view, a plurality of protrusions formed on a ∩-shaped second penile fixing portion (222) in an x-axis direction, a y-axis direction, and a z-axis direction.

FIG. 6D illustrates a plurality of protrusions formed in an x-axis direction on a surface of a ∩-shaped second penile fixing portion (222), as viewed from an opposite z-axis direction.

FIG. 7A illustrates, in a three-dimensional view, a plurality of protrusions formed on a ∩-shaped first penile fixing portion in an x-axis direction, a y-axis direction, and a z-axis direction.

FIG. 7B illustrates a plurality of protrusions formed in an opposite x-axis direction on a surface of the first penile fixing portion, as viewed from an opposite z-axis direction.

### BEST MODE FOR INVENTION

The present invention provides a urinary incontinence clamp device for men comprising: a first clamp unit comprising an inner surface formed in a ring shape and comprising a first penile fixing portion; and a second clamp unit comprising an outer surface and comprising a handle portion and a second penile fixing portion, wherein the outer surface of the second clamp unit is brought into close contact with and fixed to the inner surface of the first clamp unit, the second clamp unit is slidable along the inner surface of the first clamp unit, and the penis is fixed through the first penile fixing portion and the second penile fixing portion by utilizing magnetic force.

The present invention provides a urinary incontinence clamp device for men. The device includes a first clamp unit having an inner surface formed in a ring shape and a first penile fixing portion, and a second clamp unit having an outer surface, a handle portion, and a second penile fixing portion. The second clamp unit is configured to be in close contact with and fixed to the inner surface of the first clamp unit and to slide along the inner surface so that the penis is held between the first and second penile fixing portions.

The terms “ . . . portion,” “ . . . unit,” and the like as used in the present specification refer to a unit that processes at least one function or operation, and may be implemented in hardware, in software, or in a combination of hardware and software.

The term “ring shape” as used in the present specification may refer to a shape formed by bending a linear member and joining both ends thereof to form a rounded or angular shape, and the ring shape is not limited to a rectangular shape, a rounded shape, a circular shape, or the like. However, it is preferable that the ring shape has a form in which the second clamp unit can slide along the inner surface of the first clamp unit, which will be described later.

The linear member may include one end portion and the other end portion, and refers to a member having a linear shape in which the one end portion and the other end portion are connected, and as long as the linear member has a strength that allows the second clamp unit to slide along the inner surface of the first clamp unit, the material is not limited, for example, to steel or plastic.

The term “rectangular shape” as used in the present specification is not limited to types of quadrilaterals such as a square, a rectangle, or a rhombus.

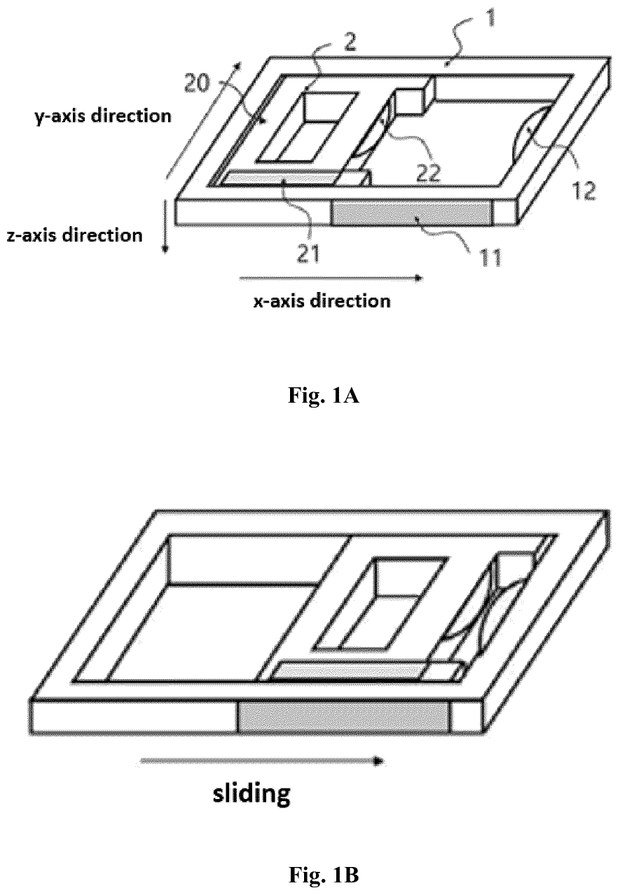

Specifically, the shape of the first clamp unit shown in FIG. 1 is an example of a rectangular ring shape, and the term “ring shape” as used in the present specification basically refers to a shape having an empty central portion.

Meanwhile, the rounded shape may be a polygonal shape having rounded corners. For example, in the case of a rectangular shape, the rounded shape may be a rectangle having its corners rounded, which may be a form in which the vertices of the rectangle are rounded. Although a rectangle is given as an example above, the polygon is not to be construed as being limited to a rectangle.

That is, the ring shape as used in the present specification is not limited as long as it has a form in which the second clamp unit can slide along the inner surface of the first clamp unit, which will be described later.

The term “inner surface” as used in the present specification may refer to an inner face of a unit having the ring shape, and the term “inner side” may refer to an inside of a unit having the ring shape.

The term “outer surface” as used in the present specification may refer to an outer face of a unit having the ring shape, and the term “outer side” may refer to an outside of a unit having the ring shape.

The term “lumen shape” as used in the present specification may refer to a shape in which an inside of a unit is hollow.

The term “x-axis direction” as used in the present specification may refer to a direction in which the second clamp unit slides along the inner surface of the first clamp unit.

The term “x-axis length” of the first clamp unit or the x-axis length of the second clamp unit as used in the present specification may refer to a length of the unit formed along a direction in which the second clamp unit slides, that is, along an x-axis direction of each clamp. For example, when the first clamp or the second clamp has a rectangular ring shape, the x-axis length may be a length of two sides formed along the x-axis direction.

The term “y-axis direction” as used in the present specification may refer to a direction perpendicular to the direction in which the second clamp unit slides along the inner surface of the first clamp unit.

The term “y-axis length” of the first clamp unit or the y-axis length of the second clamp unit as used in the present specification may refer to a length of each clamp unit formed along a direction perpendicular to the direction in which the second clamp unit slides, that is, along a y-axis direction. For example, when the first clamp or the second clamp has a rectangular ring shape, the y-axis length may be a length of two sides formed along the y-axis direction.

The term “z-axis direction” as used in the present specification may refer to a direction perpendicular to both the x-axis direction and the y-axis direction.

The term “upper portion” of the first clamp unit or the second clamp unit as used in the present specification may refer to an outer surface portion formed in a direction opposite to the x-axis direction from a center of an outer surface of the first clamp unit or the second clamp unit, and the term “lower portion” of the first clamp unit or the second clamp unit may refer to an outer surface portion formed in the x-axis direction from the center of the outer surface of the first clamp unit or the second clamp unit.

The term “central portion” of the first clamp unit or the second clamp unit as used in the present specification may refer to all portions extending from a center of gravity of the first clamp unit or the second clamp unit to an outer surface of the respective clamp, and the central portion may include a portion that is hollow in the form of a lumen.

The handle portion may be formed in the central portion of the second clamp unit in a hollow configuration in the form of a lumen, in the form of a groove in the central portion, or in any other configuration, without limitation, provided that it is in a form that allows a user to grasp the second clamp unit.

The first penile fixing portion may be positioned at a lower portion of an inner surface of the first clamp unit and, specifically, may be formed in a U-shape or an inverted U-shape (∩-shape) positioned on the inner surface of the first clamp unit formed along the y-axis direction, so as to receive and compress the penis.

The second penile fixing portion may be positioned at a lower portion of an outer surface of the second clamp unit and, specifically, may be formed in a ∩-shape (222) or a U-shape (221) positioned on the outer surface of the second clamp unit formed along the y-axis direction, so as to receive and compress the penis.

Herein, the U-shape (221) refers to a configuration in which the portion that contacts the penis is concave, such that the penis is received within the concave portion, and the ∩-shape (222) refers to a configuration in which the portion that contacts the penis is convex, such that the penis is compressed by the convex portion.

Specifically, the first penile fixing portion and the second penile fixing portion may have similar shapes, examples of which include cases where both the first penile fixing portion and the second penile fixing portion have a U-shape, and cases where both the first penile fixing portion and the second penile fixing portion have a ∩-shape.

The first penile fixing portion and the second penile fixing portion may have different shapes, examples of which include a case where the first penile fixing portion has a U-shape and the second penile fixing portion has a ∩-shape, and a case where the first penile fixing portion has a ∩-shape and the second penile fixing portion has a U-shape.

Herein, the U-shape or the ∩-shape is not limited, provided that it has a predetermined curvature sufficient to accommodate the penis.

The second penile fixing portion may be configured to compress the penis in the x-axis direction as the second clamp unit slides.

FIGS. 1A and 1B are diagrams illustrating an operational state of a urinary incontinence clamp device for men according to an embodiment of the present invention.

FIG. 1A is a diagram illustrating a state prior to sliding of the second clamp unit along an inner surface of the first clamp unit, and more particularly, a state prior to the lowering of the second penile fixing portion.

FIG. 1B is a diagram illustrating a state in which the second clamp unit has slid along an inner surface of the first clamp unit, thereby lowering the second penile fixing portion.

In the present invention, the first clamp unit comprises a first magnetic portion, and the second clamp unit comprises a second magnetic portion, wherein the first magnetic portion and the second magnetic portion may be arranged to generate an attractive force toward each other.

In the present invention, the first magnetic portion may be included in the first clamp unit and, more specifically, may be formed at a lower portion of the first clamp unit, while the second magnetic portion may be formed on an outer surface of the second clamp unit. In this case, the first magnetic portion and the second magnetic portion may generate an attractive force such that the second clamp unit slides along the inner surface of the first clamp unit, and the second clamp unit may be positioned at the lower portion of the first clamp unit so as to be arranged in parallel therewith.

The first magnetic portion and the second magnetic portion may each be formed by including a magnet within a predetermined receiving space provided in the first clamp unit and the second clamp unit, respectively. For example, the magnet may be seated and fixed within the receiving space.

The magnet may be a permanent magnet or an electromagnet, and, in terms of miniaturization or simplification of the device, it is preferable that the permanent magnet is seated and fixed within the receiving space.

The first magnetic portion may comprise a magnet having either an N-pole or an S-pole, and the second magnetic portion may also comprise a magnet having either an N-pole or an S-pole. In order for the second clamp unit to slide along the inner surface of the first clamp unit by virtue of a mutual attractive force between the first magnetic portion and the second magnetic portion, the first magnetic portion and the second magnetic portion must each have opposite polarities. For example, when the first magnetic portions positioned on both lateral sides of the first clamp unit have an N-pole, the second magnetic portions positioned on both lateral sides of the second clamp unit must have an S-pole; conversely, when the first magnetic portions positioned on both lateral sides of the first clamp unit have an S-pole, the second magnetic portions positioned on both lateral sides of the second clamp unit must have an N-pole.

The magnet may comprise a magnetic material, and the magnetic material may be selected from the group consisting of iron (Fe), copper (Cu), cobalt (Co), nickel (Ni), chromium (Cr), samarium (Sm), and aluminum (Al), and may include one or more of these materials.

The magnet included in the first magnetic portion or the second magnetic portion may be selected from the group consisting of neodymium magnets, ferrite magnets, alnico magnets, and samarium-cobalt magnets, and may comprise one or more thereof.

Male urinary incontinence is a condition that occurs when the urethral sphincter fails to withstand the discharge of urine due to a decline in the functional performance of the urethral sphincter. It is generally known that, when sneezing or coughing occurs, the pressure can increase up to 120 cmH2O.

Accordingly, in order to reliably prevent urinary incontinence, it is necessary to compress the urethra with a pressure of at least 130 cmH2O. In the present invention, by adjusting the magnetic force of the magnet so that the second penile fixing portion descends to apply a compression pressure of at least 130 cmH2O to the penis, urinary incontinence occurring even during severe sneezing or coughing can be effectively prevented.

In order to adjust to the pressure range applied to the urethra, it is necessary to regulate the magnetic force of the magnet included in the magnetic force portion and the attractive force acting between the respective magnets within a predetermined range.

The magnetic force of the magnet included in the magnetic force portion may be adjusted to a range of 3,000 to 5,000 G, thereby enabling the pressure applied to the penis to be decreased or increased. If the magnetic force of the magnet is less than 3,000 G, the pressure applied to the urethra may be insufficient, making it difficult to adequately suppress urinary incontinence. Conversely, if the magnetic force exceeds 5,000 G, the penis may be excessively compressed, causing the user to experience pain, impeding proper blood circulation, and potentially resulting in penile tissue damage or excretory dysfunction.

In continuation of the adjustment of the magnetic force, the attractive force between the magnets included in the first magnetic force portion or the second magnetic force portion may be adjusted to a range of 3.0 to 10.0 kgf, thereby enabling the pressure applied to the penis to be decreased or increased. If the attractive force is less than 3.0 kgf, the pressure applied to the urethra may be insufficient, making it difficult to adequately suppress urinary incontinence. Conversely, if the attractive force exceeds 10.0 kgf, the penis may be excessively compressed, causing the user to experience pain, impeding proper blood circulation, and potentially resulting in penile tissue damage or excretory dysfunction.

In the conventional technology in which the penis is compressed by the elastic force of an elastic material, it is not easy to adjust the compression force applied to the penis, and the elastic force of the elastic material may act instantaneously on the penis, potentially causing the user to experience pain upon wearing.

The present invention improves upon the aforementioned problems of the prior art by utilizing the magnetic force between a first clamp unit and a second clamp unit, each provided with a magnetic force portion. Specifically, as the second clamp unit is slid by the magnetic force, the force applied to the penis is gradually exerted, thereby reducing the impact force applied to the penis and enhancing wearing comfort by preventing pain during fixation.

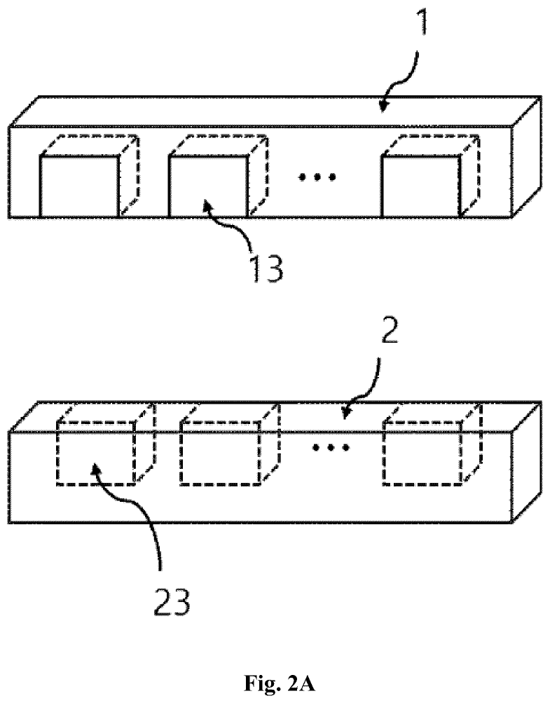

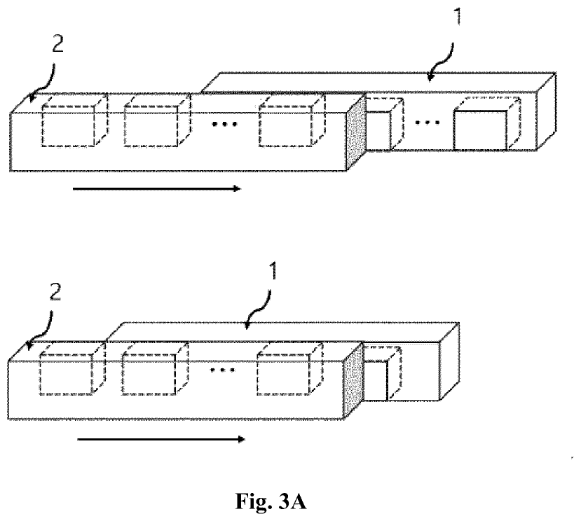

In the present invention, as shown in FIGS. 2A and 4A, the inner surface of the first clamp unit may include a single or a plurality of first retaining members, and the outer surface of the second clamp unit may include a single or a plurality of second retaining members that are structurally corresponding to the single or plurality of first retaining members.

Specifically, the inner surface of the first clamp unit is formed with a single or a plurality of first retaining members, and the outer surface of the second clamp unit may be formed with a single or a plurality of second retaining members configured to correspondingly engage with the single or plurality of first retaining members.

Herein, the term “engagement” may refer to an engagement that can occur instantaneously, but which, in the context of the movement of the second clamp unit by magnetic force, denotes a meshing engagement of corresponding portions of each member.

In one embodiment of the present invention, the first retaining member may be in the form of a retaining step or a retaining protrusion, and the second retaining member may likewise be in the form of a retaining step or a retaining protrusion. However, when the first retaining member is in the form of a retaining step, the second retaining member may be a retaining protrusion configured to engage correspondingly therewith, and when the first retaining member is in the form of a retaining protrusion, the second retaining member may be a retaining step configured to engage correspondingly therewith.

One to ten first retaining members may be formed on the inner surface of the first clamp unit, and a plurality of the second retaining members may be engaged correspondingly with the first retaining members. As the second clamp unit is slid along the inner surface of the first clamp unit by magnetic force, the engagement of the retaining protrusions with the first retaining members can mitigate the impact force applied to the penis.

The position of the second retaining member, which is correspondingly engaged with the first retaining member, may be adjustable and can be regulated according to the size of the penis by operating the handle portion. When the penile diameter is large, the position of the retaining member may be located at an upper portion of the second clamp unit, and when the penile diameter is small, the position of the retaining member may be located at a lower portion of the second clamp unit.

For example, depending on the position of the first retaining member formed on the inner surface of the first clamp unit, each first retaining member may be assigned a sequential number according to the direction in which sliding is performed, such that they may be designated as a first retaining member No. 1, a first retaining member No. 2, a first retaining member No. 3, and so on. Likewise, on the outer surface of the second clamp unit, each second retaining member may be assigned a sequential number according to the direction in which sliding is performed, such that they may be designated as a second retaining member No. 1, a second retaining member No. 2, a second retaining member No. 3, and so on. In this case, the first retaining member may be in the form of a retaining step, and the second retaining member may be in the form of a retaining protrusion.

When the second clamp unit is caused to slide by the magnetic force, the second retaining member No. 3 is first engaged with the first retaining member No. 1. When the second clamp unit is lowered via the handle, the second retaining member No. 3 may be engaged with the first retaining member No. 2.

The position at which the first retaining member and the second retaining member are engaged may be arbitrarily adjusted by the user prior to sliding of the second clamp unit. For example, in the above example, the first retaining member No. 3 and the second retaining member No. 2 may be configured to be engaged first. This allows the impact force applied to the penis by the second penile fixing portion to be mitigated in accordance with the size of the user's penis.

On the other hand, in another aspect, the first retaining member or the second retaining member may be a magnet having an N-pole or an S-pole, and the first retaining member and the second retaining member may be magnets having opposite polarities. The first retaining member and the second retaining member may be configured in a form that allows them to be engaged with each other through magnetic attraction.

Specifically, when the one or more first retaining members have an N-pole, the corresponding one or more second retaining members may have an S-pole, and when the one or more first retaining members have an S-pole, the corresponding one or more second retaining members may have an N-pole.

The inner surface of the first clamp unit may be provided with one to ten first retaining members having magnetic force, and the outer surface of the second clamp unit may be provided with one to ten second retaining members having magnetic force.

As a plurality of retaining members engage, the impact force applied to the penis during sliding of the second clamp unit along the inner surface of the first clamp unit by magnetic force can be mitigated.

The position of the second retaining member, which is configured to correspondingly engage with the first retaining member, may be adjustable according to the size of the penis by manipulation of the handle portion. When the penile diameter is large, the first retaining member may be positioned at an upper portion of the second clamp unit, and when the penile diameter is small, the retaining member may be positioned at a lower portion of the second clamp unit.

For example, depending on the position of the first retaining member formed on the inner surface of the first clamp unit, the first retaining members may be sequentially numbered in the sliding direction as “first retaining member No. 1,” “first retaining member No. 2,” “first retaining member No. 3,” and so on. Likewise, the second retaining members formed on the outer surface of the second clamp unit may be sequentially numbered in the sliding direction as “second retaining member No. 1,” “second retaining member No. 2,” “second retaining member No. 3,” and so on. The first retaining member may be a magnet having an N-pole, and the second retaining member may be a magnet having an S-pole.

When the second clamp unit is caused to slide by magnetic force, the second retaining member No.3 is first engaged with the first retaining member No. 1, and when the second clamp unit is lowered via manipulation of the handle portion, the second retaining member No.3 may be engaged with the first retaining member No.2.

The position at which the first retaining member and the second retaining member are engaged may be arbitrarily adjusted by the user prior to sliding of the second clamp unit. For example, in the above-described case, the arrangement may be set such that the first retaining member No.3 and the second retaining member No.2 are engaged first. This allows the impact force applied to the penis by the second penile fixing portion to be mitigated in accordance with the size of the user's penis.

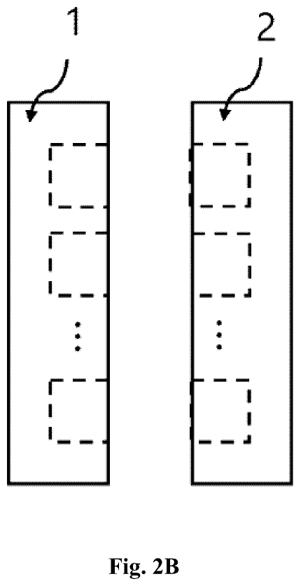

FIGS. 3A and 3B illustrate, according to one embodiment of the present invention, a state in which the first retaining member and the second retaining member are engaged and operated. In FIGS. 3A and 3B, it can be seen that, as the second clamp unit slides along the inner surface of the first clamp unit, each of the retaining members are sequentially engaged, as described above.

In the present invention, the first penile fixing portion or the second penile fixing portion may comprise an elastic material.

In the present invention, the surface of the first penile fixing portion or the second penile fixing portion may be formed of an elastic material, and the surface may be provided with a plurality of protrusions made of the elastic material.

The protrusions may be formed in various shapes, such as protrusions or embossing, and are not limited thereto, provided that they are configured to secure spaces between the protrusions when the penis is fixed to improve ventilation.

Through the plurality of protrusions, the penis can be fixed while allowing air to circulate through the gaps therebetween, thereby facilitating blood circulation of the penis compared to when no such protrusions are provided, reducing a sense of discomfort upon wearing, and preventing secretions from directly contacting the silicone, which in turn prevents deformation, loss of elasticity, or deterioration of the silicone due to the secretions.

In the present invention, the width of the protrusions may refer to the length of the protrusions formed along the z-axis direction, wherein the plurality of protrusions have a width ranging from 0.05 to 1.0 times the length in the z-axis direction. If the width of the protrusions is less than 0.05 times, a problem may arise in that sufficient space between the penis and each penile fixing portion cannot be secured, thereby impairing ventilation. Conversely, if the width exceeds 1.0 times, a problem may occur in that the width of the protrusions is formed to exceed the length of the penile fixing portion formed along the z-axis direction.

The height of the protrusions may refer to the length of the protrusions formed along the x-axis direction or in the direction opposite thereto, wherein the height of the plurality of protrusions may protrude 0.1 to 2.0 mm from the surface of the first penile fixing portion or the second penile fixing portion. If the height of the protrusions is less than 0.1 mm, a problem may arise in that sufficient space between the penis and each penile fixing portion cannot be secured, thereby impairing ventilation. Conversely, if the height exceeds 2.0 mm, a problem may occur in that fixation of the penis in the x-axis direction becomes difficult.

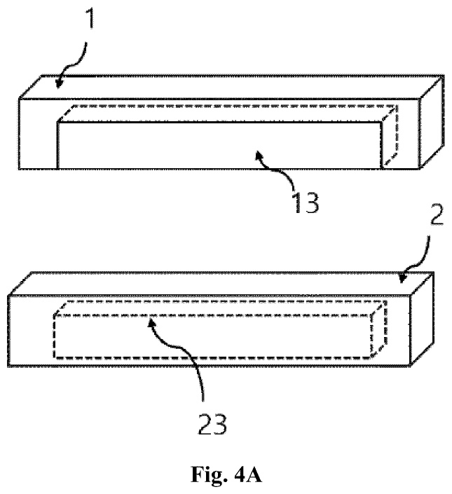

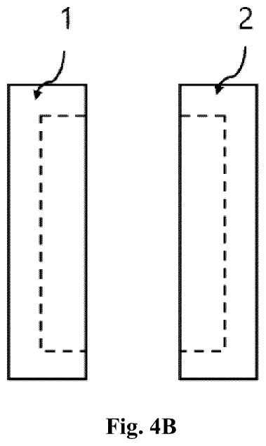

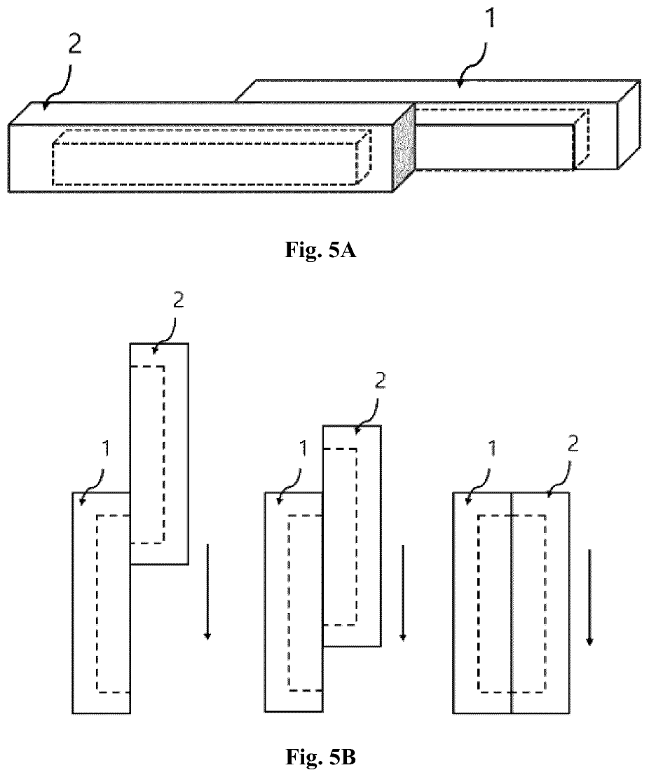

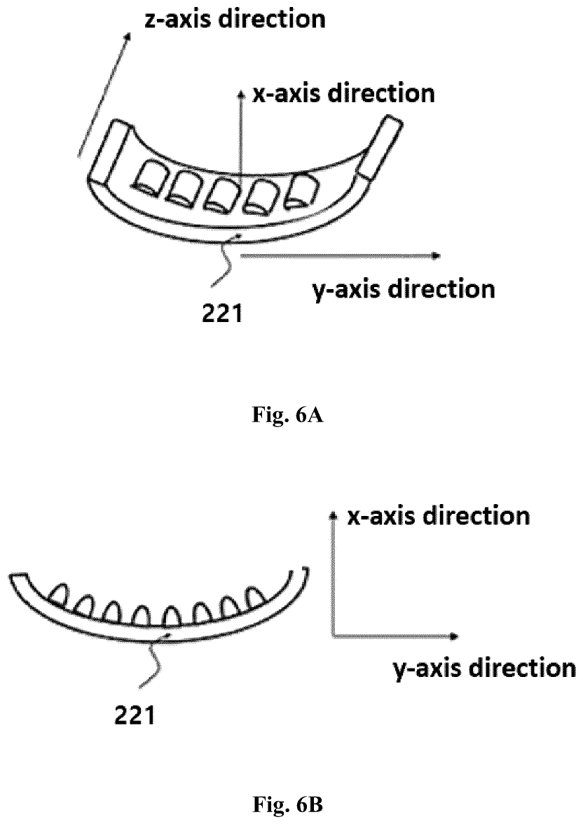

FIG. 6A is a perspective view illustrating a plurality of protrusions formed on the U-shaped second penile fixing portion in the x-axis direction, the y-axis direction, and the z-axis direction, and FIG. 6B is a view illustrating the plurality of protrusions formed in the x-axis direction on the surface of the second penile fixing portion as viewed from the reverse direction of the z-axis direction.

FIG. 6C is a perspective view illustrating a plurality of protrusions formed on the ∩-shaped second penile fixing portion in the x-axis direction, the y-axis direction, and the z-axis direction, and FIG. 6D is a view illustrating the plurality of protrusions formed in the x-axis direction on the surface of the second penile fixing portion as viewed from the reverse direction of the z-axis direction.

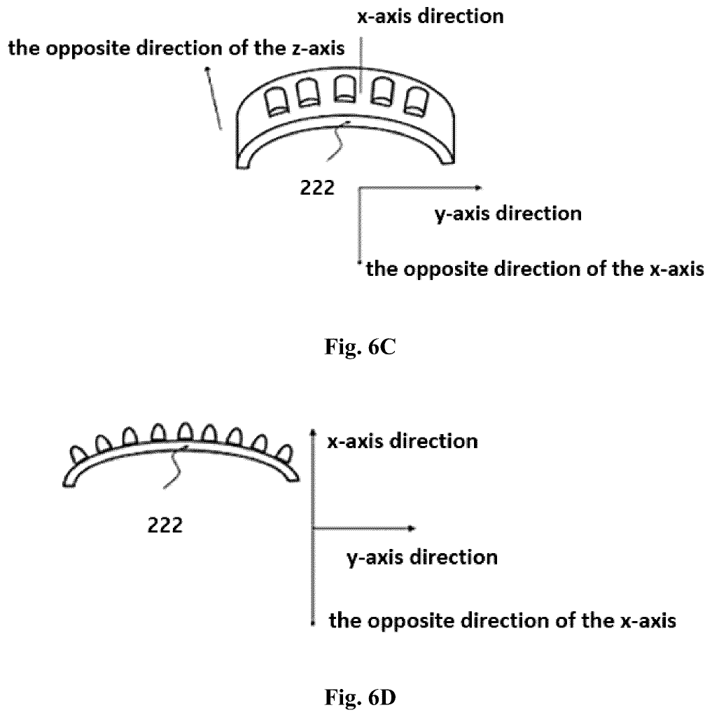

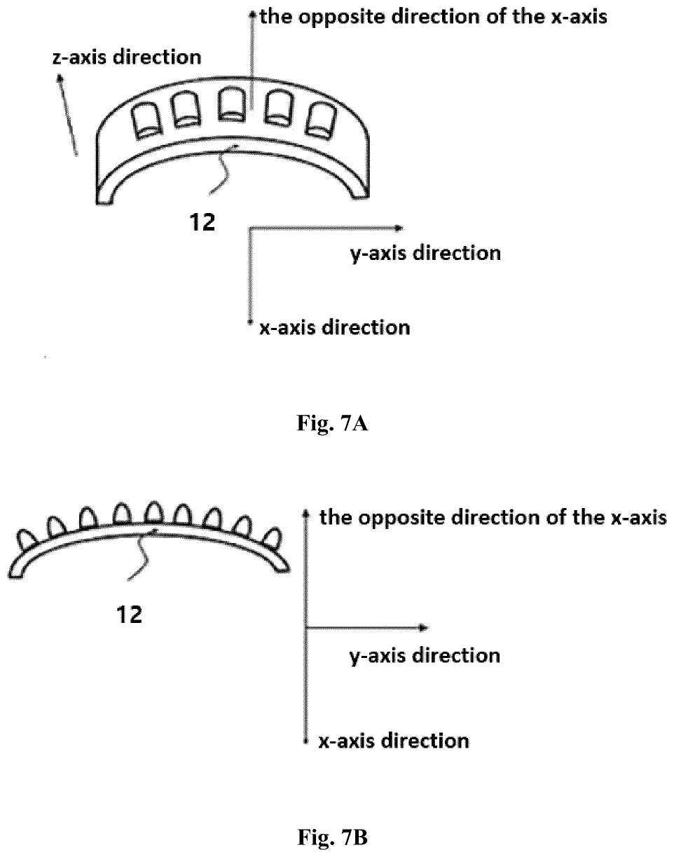

FIG. 7A is a perspective view illustrating a plurality of protrusions formed on the ∩-shaped first penile fixing portion in the x-axis direction, the y-axis direction, and the z-axis direction, and FIG. 7B is a view illustrating the plurality of protrusions formed in the reverse x-axis direction on the surface of the first penile fixing portion as viewed from the opposite direction of the z-axis direction.

The plurality of protrusions may be arranged in the y-axis direction at an interval of 0.1 to 1.0 mm. If the interval is less than 0.1 mm, the protrusions may be arranged too densely, thereby failing to secure sufficient spacing and resulting in inadequate ventilation. Conversely, if the interval exceeds 1.0 mm, there may be a problem in securing the penis in the lateral direction, and the effect resulting from the presence of the protrusions on the surface of the penile fixing portion may be diminished.

The elastic material of the surface of the first penile fixing portion or the second penile fixing portion may be an elastic material comprising silicone. Specifically, the elastic material comprising silicone may be characterized by a mixture of silicone and a rubber material, wherein the rubber material may be selected from the group consisting of acrylonitrile butadiene rubber (NBR), styrene-butadiene rubber (SBR), natural rubber (NR), polyvinyl chloride (PVC), polyethylene (PE), ethylene-vinyl acetate (EVA), latex, silicone rubber, styrene-isoprene-styrene copolymer (SIS), styrene-ethylene-butylene-styrene copolymer (SEBS), polyvinyl alcohol (PVA), butyl rubber, chloroprene rubber, and polyurethane (PU).

The weight ratio of the rubber material to the total mixture of silicone and rubber material may be in the range of 1:2 to 1:4. If the weight of the rubber material relative to the weight of the silicone is less than 2, there may be a problem in that sufficient elastic force to compress the penis and prevent urinary incontinence cannot be obtained.

Conversely, if the weight of the rubber material exceeds 4, there may be a problem in that smooth blood circulation in the penis cannot be maintained.

### DESCRIPTION OF REFERENCE NUMERALS

1: First Clamp Unit
11: First Magnetic Portion
12: First Penile Fixing Portion
13: First retaining Member
2: Second Clamp Unit
20: Handle Portion
21: Second Magnetic Portion
22: Second Penile Fixing Portion
221: U-Shaped Second Penile Fixing Portion
222: ∩-Shaped Second Penile Fixing Portion
23: Second retaining Member

### INDUSTRIAL APPLICABILITY

The present invention relates to a urinary incontinence clamp device for men, capable of adjusting the compressive force applied to the penis while improving ventilation performance, and thus can assist in the treatment process of urinary incontinence in users, thereby having industrial applicability.

1. A urinary incontinence clamp device for men, comprising:
a first clamp unit having an inner surface formed in a ring shape and including a first penile fixing portion; and
a second clamp unit having an outer surface and including a handle portion and a second penile fixing portion,
wherein the outer surface of the second clamp unit is in close contact with and fixed to the inner surface of the first clamp unit,
wherein the second clamp unit is slidable along the inner surface of the first clamp unit, such that the penis is secured between the first penile fixing portion and the second penile fixing portion,
and wherein the device operates by utilizing magnetic force.

2. The urinary incontinence clamp device for men according to claim 1,
wherein the first clamp unit comprises a first magnetic portion,
wherein the second clamp unit comprises a second magnetic portion, and
wherein the first magnetic portion and the second magnetic portion are arranged to generate an attractive force toward each other.

3. The urinary incontinence clamp device for men according to claim 1,
wherein the inner surface of the first clamp unit comprises one or more first retaining members, and
wherein the outer surface of the second clamp unit comprises one or more second retaining members structurally corresponding to the one or more first retaining members.

4. The urinary incontinence clamp device for men according to claim 2,
wherein the first magnetic portion comprises one or more first retaining members, and
wherein the second magnetic portion comprises one or more second retaining members structurally corresponding to the one or more first retaining members.

5. The urinary incontinence clamp device for men according to claim 1,
wherein a surface of the first penile fixing portion or the second penile fixing portion is made of an elastic material, and
wherein the surface is formed with a plurality of protrusions made of the elastic material.

6. The urinary incontinence clamp device for men according to claim 5,
wherein a width of the plurality of protrusions have a width ranging from 0.05 to 1.0 times a length in a z-axis direction.

7. The urinary incontinence clamp device for men according to claim 5,
wherein the plurality of protrusions project from the surface in a direction along the x-axis or opposite to the x-axis by 0.1 to 2.0 mm.

8. The urinary incontinence clamp device for men according to claim 5,
wherein the elastic material comprises silicone.

9. The urinary incontinence clamp device for men according to claim 8,
wherein the elastic material comprising silicone is characterized by a mixture of silicone and a rubber material,
and the rubber material is at least one selected from the group consisting of acrylonitrile butadiene rubber (NBR), styrene-butadiene rubber (SBR), natural rubber (NR), polyvinyl chloride (PVC), polyethylene (PE), ethylene-vinyl acetate (EVA), latex, silicone rubber, styrene-isoprene-styrene (SIS) copolymer, styrene-ethylene-butylene-styrene (SEBS) copolymer, polyvinyl alcohol (PVA), butyl rubber, chloroprene rubber, and polyurethane (PU).
